(19) United States
(12) Patent Application Publication
(10) Pub. No.: US 2026/0021253 A1
(43) Pub. Date: Jan. 22, 2026

(54) Flat Syringe

(71) Applicants: Thomas J. Shaw, Frisco, TX (US); Retractable Technologies, Inc., Little Elm, TX (US)

(72) Inventors: Thomas J. Shaw, Frisco, TX (US); Benjamin John Lardas, Denton, TX (US)

(21) Appl. No.: 19/271,372

(22) Filed: Jul. 16, 2025

Related U.S. Application Data
(60) Provisional application No. 63/672,527, filed on Jul. 17, 2024.

(51) Int. Cl. A61M 5/31; A61M 5/315

(52) U.S. Cl. CPC A61M 5/3137; A61M 5/31513; A61M 2005/314

(57) ABSTRACT

Systems and methods are disclosed that include providing a flat syringe having a substantially flat barrel having a main body portion with substantially flat upper and lower surfaces. The main body portion defines a fluid chamber through the barrel and includes a plunger assembly having a plunger seal that slidably engages the fluid chamber to form a fluid tight seal between the plunger assembly and the barrel. The barrel comprises a width (W_{B}) that is greater than the thickness (T_{B}) of the barrel to define the flat profile of the barrel and the syringe. Each of the barrel and the fluid chamber have substantially rounded or beveled rectangular cross-sectional shapes that further define the flat profile of the barrel and the syringe.

### CROSS-REFERENCE TO RELATED APPLICATIONS

This application claims priority under 35 U.S.C. § 119 (c) to U.S. Provisional Patent Application No. 63/672,527 filed on Jul. 17, 2024 by Thomas J. Shaw et. al. and entitled “Flat Syringe,” the disclosure of which is hereby incorporated by reference in its entirety for all purposes.

### BACKGROUND

Medical syringes are used for administering injections or infusions of drugs, medicines, and vaccines all over the world. Various types of injections typically require different depths and/or different angles of penetration into the patient. For example, intravenous injections often require extreme angles to avoid penetrating through a vein and rupturing a vein. Precision injections such as botulinum neurotoxin injections that are administered intradermally, often require extreme angles to ensure the desired tissue is treated. Administering such injections presents challenges to medical professionals, where traditional syringes are often too bulky to achieve these desired angles and may contact or interfere with the injection site when attempting to achieve these desired angles. Thus, the medical industry continues to demand advances in syringe technology that aid the user in administering the injection at a desired angle without the interference caused by bulky traditional syringes, thereby increasing confidence and safety for both the patient and the user.

### SUMMARY

Embodiments of a flat syringe disclosed herein include providing the flat syringe with a substantially flat barrel having a main body portion with substantially flat upper and lower surfaces. The main body portion defines a fluid chamber through the barrel and includes a plunger assembly having a plunger seal that slidably engages the fluid chamber to form a fluid tight seal between the plunger assembly and the barrel. The barrel comprises a width (W_{B}) that is greater than the thickness (T_{B}) of the barrel to define the flat profile of the barrel and the syringe. Each of the barrel and the fluid chamber have substantially rounded or beveled rectangular cross-sectional shapes that further define the flat profile of the barrel and the syringe. An optional needle may be disposed within a needle holder and connected in fluid communication with the fluid chamber for administration of an injection.

Embodiments of a flat syringe disclosed herein include: a substantially flat barrel including a main body portion that defines a fluid chamber and an annularly projecting finger flange disposed at a rear end of the main body portion, wherein the barrel includes a width (W_{B}) and a thickness (T_{B}), and wherein the width is greater than the thickness; and a plunger assembly disposed within the barrel and including a plunger seal that slidably engages the fluid chamber to form a fluid tight seal between the plunger assembly and the barrel.

Embodiments of a flat syringe disclosed herein include: a substantially flat barrel including a main body portion that defines a fluid chamber, an annularly projecting finger flange disposed at a rear end of the main body portion, and a needle holder projecting forwardly from a nose end of the main body portion, wherein the barrel includes a width (W_{B}) and a thickness (T_{B}), and wherein the width is greater than the thickness; a needle disposed in a needle aperture of the needle holder and projecting forwardly from the needle holder, wherein the needle is connected in fluid communication with the fluid chamber; and a plunger assembly disposed within the barrel and including a plunger seal that slidably engages the fluid chamber to form a fluid tight seal between the plunger assembly and the barrel.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique view of a flat syringe according to an embodiment of the disclosure.

FIG. 2 shows an oblique exploded view of the flat syringe according to an embodiment of the disclosure.

FIG. 3 shows an orthogonal top view of the flat syringe according to an embodiment of the disclosure.

FIG. 4 shows a cross-sectional front view of the flat syringe according to an embodiment of the disclosure.

FIG. 5 shows a partial cross-sectional side view of a front portion of the flat syringe according to an embodiment of the disclosure.

The use of the same reference symbols in different drawings indicates similar or identical items.

### DETAILED DESCRIPTION

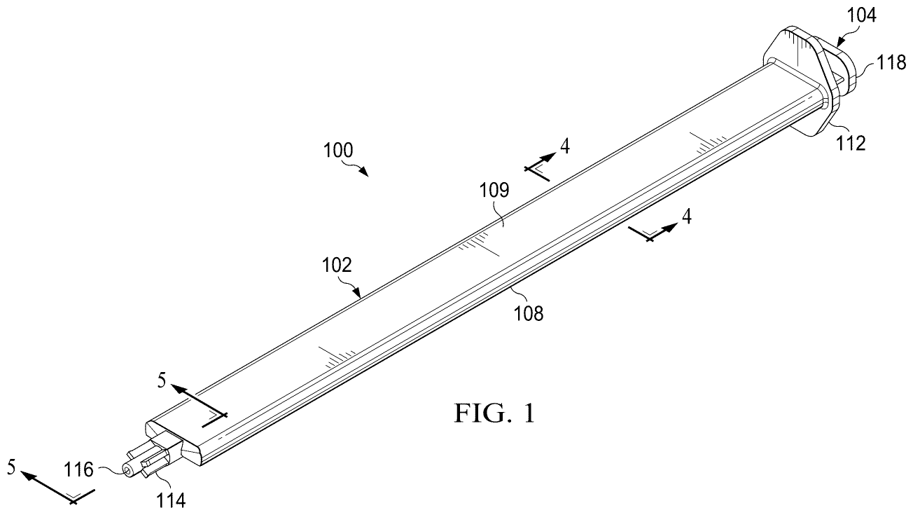

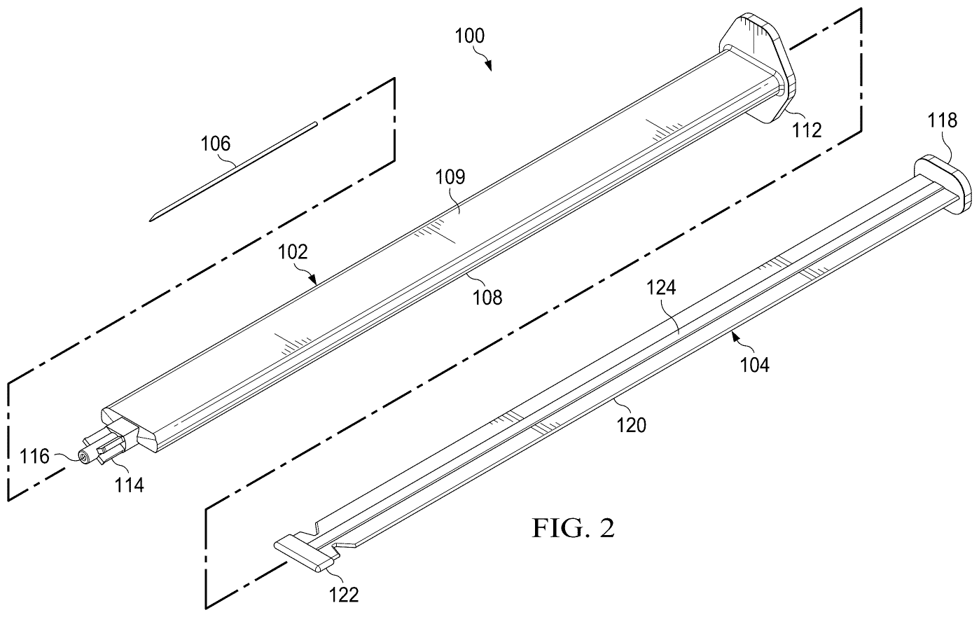

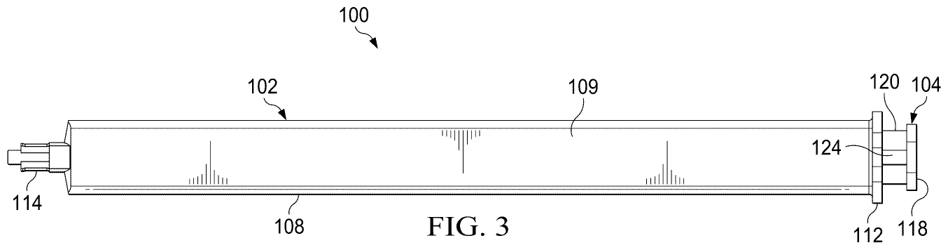

Referring to FIGS. 1 to 3, an oblique view, an oblique exploded view, an orthogonal top view, and a partial cross-sectional side view of a flat syringe 100 are shown according to an embodiment of the disclosure. The syringe 100 may generally comprise a flat barrel 102 and a plunger assembly 104 slidably disposed within the barrel 102. In some embodiments, the syringe 100 may also comprise a needle 106. The barrel 102 may generally comprise a main body portion 108 that defines a fluid chamber 110 within the barrel 102. The main body portion 108 may comprise substantially flat upper and lower surfaces 109. The barrel 102 may also comprise an annularly projecting finger flange 112 disposed at the rear end of the barrel 102 and/or the main body portion 108. The barrel 102 may comprise a needle holder 114 disposed at a nose end or forward end of the main body portion 108 for receiving and securing the needle 106 to the barrel 102. In some embodiments, the needle holder 114 may project forwardly from the barrel 102 and comprise a needle aperture 116 for receiving the needle 106 and connecting the needle 106 in fluid communication with the fluid chamber 110 of the barrel 102.

In some embodiments, the needle 106 may be secured within the needle aperture 116 of the barrel 102 via friction and/or an interference fit. However, in some embodiments, the needle 106 may be secured within the needle aperture 116 of the barrel 102 via a medical grade adhesive. In alternative embodiments, the needle holder 114 may comprise a separate component that is threadably attachable or frictionally engageable with the barrel 102 to secure the needle 106 to the barrel 102. In some embodiments, the needle 106 may be pre-installed into the needle holder 114 during manufacturing and/or prior to shipment. However, in some embodiments, the needle 106 may be inserted into the needle holder 114 by a medical professional or user prior to administering an injection.

The plunger assembly 104 may be inserted forwardly through a rear opening in the barrel 102 and into sliding engagement with the fluid chamber 110 of the main body portion 108 of the barrel 102. The plunger assembly 104 may generally comprise a plunger surface 118 for selectively actuating or manipulating the plunger assembly 104 within the barrel 102, an elongated plunger body 120 extending forwardly therefrom, and a plunger seal 122 disposed at a distal end of the plunger body 120. The plunger body 120 may be at least partially received within the main body portion 108 of the barrel 102, such that the plunger seal 122 forms a fluid tight seal between plunger assembly 104 and the barrel 102, and more specifically, between the plunger seal 122 and the fluid chamber 110 formed by the main body portion 108 of the barrel 102.

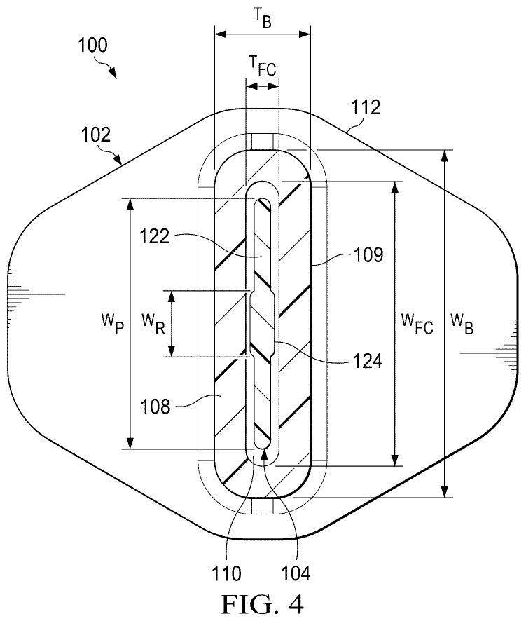

The plunger body 120 may generally extend from the plunger surface 118 to a distal end portion comprising the plunger seal 122. In some embodiments, the plunger body 120 may comprise a spine or rib 124 extending from the plunger surface 118 and longitudinally along the plunger body. In some embodiments, the rib 124 may extend from the plunger surface 118 to the plunger seal 122. In some embodiments, the rib 124 may be configured to provide structural rigidity to the plunger body 120, and consequently the plunger assembly 104. In some embodiments, the rib 124 may comprise a width (W_{R}) that is at least 10%, at least 15%, at least 20%, or at least 25% of the width (W_{P}) of the plunger body 120 as shown in FIG. 4.

The plunger seal 122 may slidably contact the inner surfaces of the fluid chamber 110 of the barrel 102 to form a fluid tight seal between the plunger assembly 104 and the barrel 102. In some embodiments, the plunger seal 122 may be integrally formed with the plunger body 120. In some embodiments, the plunger seal 122 may be formed from a medical grade thermoplastic material as the plunger body 120 and remainder of the plunger assembly 104 when integrally formed. In some embodiments, the plunger seal 122 may be overmolded over a distal end projection or distal end portion of the plunger body 120. In some embodiments, the plunger seal 122 may be formed from a polymeric material having a low coefficient of friction when overmolded. In some embodiments, the plunger seal 122 may comprise a separate component that is disposed on or over a distal end projection or distal end portion of the plunger body 120. In some embodiments, the plunger seal 122 may be formed from an elastomeric material when overmolded or comprising a separate component.

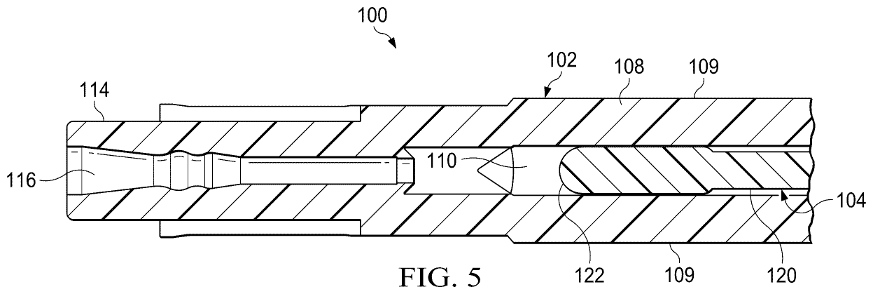

Referring specifically to FIGS. 4 and 5, a cross-sectional front view and a partial cross-sectional side view of a front portion of the flat syringe 100 are shown according to an embodiment of the disclosure. In some embodiments, the barrel 102 may comprise a rounded or beveled rectangular cross-sectional shape having a width (W_{B}) that spans the substantially flat surfaces 109 of the main body portion 108 of the barrel 102 and a thickness (T_{B}). In some embodiments, the fluid chamber 110 may also comprise a substantially similar rounded or beveled rectangular cross-sectional shape having a width (W_{FC}) and a thickness (T_{FC}). In some embodiments, the fluid chamber 110 may comprise a substantially rectangular cross-sectional profile having rounded ends. In some embodiments, the rounded ends of the fluid chamber 110 may enhance the fluid tight seal between the fluid chamber 110 and the plunger seal 122. As such, it will be appreciated that the plunger seal 122 may comprise a substantially similar cross-sectional profile as the fluid chamber 110. However, in other embodiments, the barrel 102 and/or the fluid chamber 110 may comprise any other suitable cross-sectional profile, such as rectangular or oval.

The barrel 102 may generally comprise a substantially slim or flat profile. In some embodiments, the substantially slim or flat profile may be at least partially defined by the substantially flat surfaces 109 of the main body portion 108 of the barrel 102. The flat profile of the barrel indicates that the barrel 102 comprises a width (W_{B}) that is larger than the thickness (T_{B}). The larger width (W_{B}) of the barrel 102 may provide the fluid chamber 110 with an adequate volume inside the barrel 102 for delivering a requisite dosage of an injection. For example, in some embodiments, the fluid chamber 110 of the barrel 102 may comprise a volume of at least 0.25 milliliters (mL), at least 0.50 mL, at least 0.75 mL, at least 1.0 mL, at least 2 mL, or even at least 3 mL. Further, the larger width (W_{B}) of the barrel 102 may also provide structural rigidity to the barrel 102, and consequently the syringe 100, to prevent bending or deformation of the barrel 102 and/or syringe 100 during administration of an injection.

In some embodiments, the barrel 102 may comprise a width to thickness ratio (W_{B}:T_{B}) that defines the flatness of the barrel 102. In some embodiments, the width to thickness ratio (W_{B}:T_{B}) of the barrel 102 may be at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, or even at least 5:1. Further, in some embodiments, the width to thickness ratio (W_{B}:T_{B}) of the barrel 102 may be any of these absolute values, such as about 3.5:1, or in a range of these values, such as at least 2:1 to not greater than 4:1.

The fluid chamber 110 may comprise a substantially similar or complementary cross-sectional shape to the barrel 102. Accordingly, the fluid chamber 110 may also comprise a width to thickness ratio (W_{FC}:T_{FC}) that defines the flatness of the barrel 102 and/or the fluid chamber 110. In some embodiments, the width to thickness ratio (W_{FC}:T_{FC}) of the fluid chamber 110 may be at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, at least 5:1, at least 6:1, at least 7:1, at least 8:1, at least 9:1, or even at least 10:1. Further, in some embodiments, the width to thickness ratio (W_{FC}:T_{FC}) of the fluid chamber 110 may be any of these absolute values, such as about 8:1, or in a range of these values, such as at least 4:1 to not greater than 10:1.

In some embodiments, the syringe 100 may also comprise any combination of these width to thickness ratio values for each of the barrel 102 and the fluid chamber 110. For example, the flat syringe 100 may comprise a width to thickness ratio (W_{B}:T_{B}) of the barrel 102 of about 3.5:1 and width to thickness ratio (W_{FC}:T_{FC}) of the fluid chamber 110 of about 8:1. In another example, the flat syringe 100 may comprise a width to thickness ratio (W_{B}:T_{B}) of the barrel 102 of at least 2:1 to not greater than 4:1 and width to thickness ratio (W_{FC}:T_{FC}) of the fluid chamber 110 of at least 4:1 to not greater than 10:1.

By providing the syringe 100 with a flat barrel 102, the syringe 100 may be referred to as a flat syringe 100. The flatness of the barrel 102 may allow extreme angles for an injection, where the barrel 102 may get extremely close to the injection site or the skin surface of a patient. In some embodiments, the flat barrel 102 may permit injections at an angle of less than 45 degrees, less than 30 degrees, less than 25 degrees, less than 20 degrees, less than 15 degrees, less than 10 degrees, less than 5 degrees, less than 2.5 degrees, or even lower with respect to the injection site or skin surface of a patient. The flatness of the barrel 102 may also allow the syringe 100 to be inserted into a narrow incision for injection into an organ or deeper tissue of a patient to enable a minimally invasive injection that may be needed beneath the surface of the skin or underneath a bone or organ. Further, while the syringe 100 is disclosed with respect to giving an injection, the syringe 100 may also be applicable to blood draws, biopsy samples, or other withdrawals of a bodily fluid or specimen from a patient. Still further, while the flat barrel 102 is disclosed in the context of syringe 100, the flat barrel 102 may also be applicable to blood collection tube holders, catheters, or other medical devices requiring insertion of a needle at extreme angles or where access to the injection site may be obstructed or restricted.

In some embodiments, a flat syringe or a syringe with a substantially flat barrel main body are particularly well suited for injections on facial areas and/or for injections of certain types of injectable medicines or medical preparations that are used for various medical or cosmetic treatments. Such medicines or medical preparations may comprise botulinum toxin type A, onabotulinumtoxinA (such as Botox®), abobotulinumtoxinA (such as Dysport®), incobotulinumtoxinA (such as Xeomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.

### Examples

It will be appreciated that Examples of a flat syringe 100 may include one or more of the following Examples:

Example 1. A syringe, comprising: a barrel comprising a main body portion that defines a fluid chamber and a finger flange that annularly projects and is disposed at a rear end of the main body portion, wherein the main body portion is substantially flat, wherein the barrel comprises a width (W_{B}) and a thickness (T_{B}), and wherein the width is greater than the thickness; and a plunger assembly disposed within the barrel and comprising (1) a plunger seal that slidably engages the fluid chamber to form a fluid tight seal between the plunger assembly and the barrel and (2) a plunger body.

Example 2. The syringe of any of the preceding Examples, wherein the main body portion comprises substantially flat upper and lower surfaces.

Example 3. The syringe of any of the preceding Examples, wherein the barrel comprises a needle holder projecting forwardly from a nose end of the main body portion of the barrel.

Example 4. The syringe of any of the preceding Examples, further comprising: a needle disposed in a needle aperture of the needle holder and projecting forwardly from the needle holder.

Example 5. The syringe of any of the preceding Examples, wherein the needle is connected in fluid communication with the fluid chamber.

Example 6. The syringe of any of the preceding Examples, wherein the main body portion, the finger flange, and the needle holder are integrally formed to form a unitarily molded, one-piece barrel.

Example 7. The syringe of any of the preceding Examples, wherein the needle holder is threadably attachable or frictionally engageable with the main body portion of the barrel.

Example 8. The syringe of any of the preceding Examples, wherein the plunger assembly comprises a plunger surface for selectively actuating the plunger assembly and an elongated plunger body extending from the plunger surface, wherein the plunger seal is disposed at a distal end of the plunger body.

Example 9. The syringe of any of the preceding Examples, wherein the plunger body comprises a rib extending from the plunger surface and longitudinally along the plunger body.

Example 10. The syringe of any of the preceding Examples, wherein the rib comprises a width (W_{R}) that is at least 10%, at least 15%, at least 20%, or at least 25% of a width (W_{P}) of the plunger body.

Example 11. The syringe of any of the preceding Examples, wherein the plunger seal is integrally formed with the plunger body.

Example 12. The syringe of any of the preceding Examples, wherein the plunger seal is formed from a medical grade thermoplastic material.

Example 13. The syringe of any of the preceding Examples, wherein the plunger seal is overmolded over the distal end of the plunger body.

Example 14. The syringe of any of the preceding Examples, wherein the plunger seal is formed from a polymeric material having a low coefficient of friction or an elastomeric material.

Example 15. The syringe of any of the preceding Examples, wherein the plunger seal comprises a separate component and is disposed on the distal end of the plunger body.

Example 16. The syringe of any of the preceding Examples, wherein the plunger seal is formed from an elastomeric material.

Example 17. The syringe of any of the preceding Examples, wherein the barrel comprises a rounded or beveled rectangular cross-sectional shape.

Example 18. The syringe of any of the preceding Examples, wherein the barrel comprises a width to thickness ratio (W_{B}:T_{B}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, or even at least 5:1.

Example 19. The syringe of any of the preceding Examples, wherein the width to thickness ratio (W_{B}:T_{B}) is at least 1.05:1 to not greater than 5:1.

Example 20. The syringe of any of the preceding Examples, wherein the width to thickness ratio (W_{B}:T_{B}) defines the flatness of the barrel.

Example 21. The syringe of any of the preceding Examples, wherein the fluid chamber comprises a rounded or beveled rectangular cross-sectional shape having a width (W_{FC}) and a thickness (T_{FC}).

Example 22. The syringe of any of the preceding Examples, wherein the fluid chamber comprises a substantially rectangular cross-sectional profile having rounded ends.

Example 23. The syringe of any of the preceding Examples, wherein the fluid chamber comprises a width to thickness ratio (W_{FC}:T_{FC}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, at least 5:1, at least 6:1, at least 7:1, at least 8:1, at least 9:1, or even at least 10:1.

Example 24. The syringe of any of the preceding Examples, wherein the width to thickness ratio (W_{FC}:T_{FC}) is at least 1.05:1 to not greater than 10:1.

Example 25. The syringe of any of the preceding Examples, wherein the width to thickness ratio (W_{B}:T_{B}) defines the flatness of the barrel.

Example 26. The syringe of any of the preceding Examples, wherein the fluid chamber comprises a volume of at least 0.25 milliliters (mL), at least 0.50 mL, at least 0.75 mL, at least 1.0 mL, at least 2 mL, or even at least 3 mL.

Example 27. The syringe of any of the preceding Examples, wherein the barrel comprises a width to thickness ratio (W_{B}:T_{B}) of at least 1.05:1 to not greater than 5:1, and wherein the fluid chamber comprises a width to thickness ratio (W_{FC}:T_{FC}) of at least 1.05:1 to not greater than 10:1.

Example 28. The syringe of any of the preceding Examples, wherein the barrel allows injections at an angle of less than 45 degrees, less than 30 degrees, less than 25 degrees, less than 20 degrees, less than 15 degrees, less than 10 degrees, less than 5 degrees, less than 2.5 degrees, or even lower with respect to an injection site or skin surface of a patient.

Example 29. A medical syringe, comprising: a substantially flat barrel comprising a main body portion that defines a fluid chamber, an annularly projecting finger flange disposed at a rear end of the main body portion, and a needle holder projecting forwardly from a nose end of the main body portion, wherein the barrel comprises a width (W_{B}) and a thickness (T_{B}), and wherein the width is greater than the thickness; a needle disposed in a needle aperture of the needle holder and projecting forwardly from the needle holder, wherein the needle is connected in fluid communication with the fluid chamber; and a plunger assembly disposed within the barrel and comprising a plunger seal that slidably engages the fluid chamber to form a fluid tight seal between the plunger assembly and the barrel.

Example 30. The medical syringe of any of the preceding Examples, wherein the main body portion comprises substantially flat upper and lower surfaces.

Example 31. The medical syringe of any of the preceding Examples, wherein the main body portion, the finger flange, and the needle holder are integrally formed to form a unitarily molded, one-piece barrel.

Example 32. The medical syringe of any of the preceding Examples, wherein the needle holder is threadably attachable or frictionally engageable with the main body portion of the barrel.

Example 33. The medical syringe of any of the preceding Examples, wherein the plunger assembly comprises a plunger surface for selectively actuating the plunger assembly and an elongated plunger body extending from the plunger surface, wherein the plunger seal is disposed at a distal end of the plunger body.

Example 34. The medical syringe of any of the preceding Examples, wherein the plunger body comprises a rib extending from the plunger surface and longitudinally along the plunger body.

Example 35. The medical syringe of any of the preceding Examples, wherein the rib comprises a width (W_{R}) that is at least 10%, at least 15%, at least 20%, or at least 25% of a width (W_{P}) of the plunger body.

Example 36. The medical syringe of any of the preceding Examples, wherein the plunger seal is integrally formed with the plunger body.

Example 37. The medical syringe of any of the preceding Examples, wherein the plunger seal is formed from a medical grade thermoplastic material.

Example 38. The medical syringe of any of the preceding Examples, wherein the plunger seal is overmolded over the distal end of the plunger body.

Example 39. The medical syringe of any of the preceding Examples, wherein the plunger seal is formed from a polymeric material having a low coefficient of friction or an elastomeric material.

Example 40. The medical syringe of any of the preceding Examples, wherein the plunger seal comprises a separate component and is disposed on the distal end of the plunger body.

Example 41. The medical syringe of any of the preceding Examples, wherein the plunger seal is formed from an elastomeric material.

Example 42. The medical syringe of any of the preceding Examples, wherein the barrel comprises a rounded or beveled rectangular cross-sectional shape.

Example 43. The medical syringe of any of the preceding Examples, wherein the barrel comprises a width to thickness ratio (W_{B}:T_{B}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, or even at least 5:1.

Example 44. The medical syringe of any of the preceding Examples, wherein the width to thickness ratio (W_{B}:T_{B}) is at least 1.05:1 to not greater than 5:1.

Example 45. The medical syringe of any of the preceding Examples, wherein the width to thickness ratio (W_{B}:T_{B}) defines the flatness of the barrel.

Example 46. The medical syringe of any of the preceding Examples, wherein the fluid chamber comprises a rounded or beveled rectangular cross-sectional shape having a width (W_{FC}) and a thickness (T_{FC}).

Example 47. The medical syringe of any of the preceding Examples, wherein the fluid chamber comprises a substantially rectangular cross-sectional profile having rounded ends.

Example 48. The medical syringe of any of the preceding Examples, wherein the fluid chamber comprises a width to thickness ratio (W_{FC}:T_{FC}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, at least 5:1, at least 6:1, at least 7:1, at least 8:1, at least 9:1, or even at least 10:1.

Example 49. The medical syringe of any of the preceding Examples, wherein the width to thickness ratio (W_{FC}:T_{FC}) is at least 1.05:1 to not greater than 10:1.

Example 50. The medical syringe of any of the preceding Examples, wherein the width to thickness ratio (W_{B}:T_{B}) defines the flatness of the barrel.

Example 51. The medical syringe of any of the preceding Examples, wherein the fluid chamber comprises a volume of at least 0.25 milliliters (mL), at least 0.50 mL, at least 0.75 mL, at least 1.0 mL, at least 2 mL, or even at least 3 mL.

Example 52. The medical syringe of any of the preceding Examples, wherein the barrel comprises a width to thickness ratio (W_{B}:T_{B}) of at least 1.05:1 to not greater than 5:1, and wherein the fluid chamber comprises a width to thickness ratio (W_{FC}:T_{FC}) of at least 1.05:1 to not greater than 10:1.

Example 53. The medical syringe of any of the preceding Examples, wherein the barrel allows injections at an angle of less than 45 degrees, less than 30 degrees, less than 25 degrees, less than 20 degrees, less than 15 degrees, less than 10 degrees, less than 5 degrees, less than 2.5 degrees, or even lower with respect to an injection site or skin surface of a patient.

Example 54. A method of administering an injection using a syringe according to any one of Examples 1-28 that comprises a needle or a medical syringe according to any one of Examples 29-53, the method comprising: drawing a medicine or medical preparation through the needle into the fluid chamber; positioning the syringe or the medical syringe in a desired location on a patient to receive the injection; inserting the needle into a patient; and injecting a fluid from the fluid chamber through the needle into the patient.

Example 55. The method of Example 54, further comprising adjusting an angle of the syringe or the medical syringe to allow the inserting of the needle at an angle of less than 45 degrees.

Example 56. The method of any one of Examples 54-55 wherein the fluid comprises botulinum toxin type A, onabotulinumtoxinA (such as Botox®), abobotulinumtoxinA (such as Dysport®), incobotulinumtoxinA (such as Xeomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Any structural elements or arrangement of structural elements relative to each other, or method steps, or medicine or medical preparation, of an embodiment, or a preferred embodiment, herein may be used with any other structural elements, features, components, medicines, medical preparations, or steps of other embodiments even if not specifically described with respect to that embodiment, unless such combination is explicitly excluded herein. Any structural element or arrangement of structural elements relative to each other, or method steps, or medicine or medical preparation of an embodiment described as excluded with any particular embodiment, or preferred embodiment, herein may similarly be excluded with any other embodiment herein even if not specifically described with such embodiment. Further, references to values or ratios stated in ranges include each and every individual value or ratio within that range and include sub-ranges that overlap from one preferred range to another preferred range and even if the specific subset of the range is not specifically described herein. Angles expressed as a range of “less than” a specific angle include angles between 0° and the stated angle. For example, a range of less than 45° means 0° to 45° and any individual angle or subrange of angles therebetween.

What is claimed is:

1. A syringe, comprising:
a substantially flat barrel comprising a main body portion that defines a fluid chamber and an annularly projecting finger flange disposed at a rear end of the main body portion, wherein the substantially flat barrel comprises a width (W_{B}) and a thickness (T_{B}), and wherein the width is greater than the thickness; and
a plunger assembly disposed within the substantially flat barrel and comprising a plunger body and a plunger seal that slidably engages the fluid chamber to form a fluid tight seal between the plunger assembly and the substantially flat barrel.

2. The syringe of claim 1, wherein the main body portion comprises substantially flat upper and lower surfaces.

3. The syringe of claim 1, wherein the substantially flat barrel comprises a needle holder projecting forwardly from a nose end of the main body portion of the substantially flat barrel.

4. The syringe of claim 3, further comprising: a needle disposed in a needle aperture of the needle holder and projecting forwardly from the needle holder.

5. The syringe of claim 4, wherein the needle is connected in fluid communication with the fluid chamber.

6. The syringe of claim 5, wherein the main body portion, the annularly projecting finger flange, and the needle holder are integrally formed to form a unitarily molded, one-piece barrel.

7. The syringe of claim 1, wherein the plunger assembly comprises a plunger surface for selectively actuating the plunger assembly and an elongated plunger body extending from the plunger surface, wherein the plunger seal is disposed at a distal end of the plunger body.

8. The syringe of claim 7, wherein the plunger body comprises a rib extending from the plunger surface and longitudinally along the plunger body.

9. The syringe of claim 8, wherein the rib comprises a width (W_{R}) that is at least 10%, at least 15%, at least 20%, or at least 25% of a width (W_{P}) of the plunger body.

10. The syringe of claim 7, wherein the plunger seal is integrally formed with the plunger body, overmolded over the distal end of the plunger body, or comprises a separate component and is disposed on the distal end of the plunger body.

11. The syringe of claim 10, wherein the plunger seal is formed from an elastomeric material.

12. The syringe of claim 1, wherein the substantially flat barrel comprises a rounded or beveled rectangular cross-sectional shape.

13. The syringe of claim 12, wherein the substantially flat barrel comprises a width to thickness ratio (W_{B}:T_{B}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, or even at least 5:1.

14. The syringe of claim 13, wherein the width to thickness ratio (W_{B}:T_{B}) is at least 1.05:1 to not greater than 5:1.

15. The syringe of claim 1, wherein the fluid chamber comprises a rounded or beveled rectangular cross-sectional shape having a width (W_{FC}) and a thickness (T_{FC}).

16. The syringe of claim 15, wherein the fluid chamber comprises a substantially rectangular cross-sectional profile having rounded ends.

17. The syringe of claim 15, wherein the fluid chamber comprises a width to thickness ratio (W_{FC}:T_{FC}) of at least 1.05:1, at least 1.10:1, at least 1.15:1, at least 1.20:1, at least 1.25:1, at least 1.5:1, at least 1.75:1, at least 2:1, at least 2.5:1, at least 3:1, at least 3.5:1, at least 4:1, at least 5:1, at least 6:1, at least 7:1, at least 8:1, at least 9:1, or even at least 10:1.

18. The syringe of claim 17, wherein the width to thickness ratio (W_{FC}:T_{FC}) is at least 1.05:1 to not greater than 10:1.

19. The syringe of claim 1, wherein the substantially flat barrel comprises a width to thickness ratio (W_{B}:T_{B}) of at least 1.05:1 to not greater than 5:1, and wherein the fluid chamber comprises a width to thickness ratio (W_{FC}:T_{FC}) of at least 1.05:1 to not greater than 10:1.

20. The syringe of claim 1, wherein the substantially flat barrel allows injections at an angle of less than 45 degrees, less than 30 degrees, less than 25 degrees, less than 20 degrees, less than 15 degrees, less than 10 degrees, less than 5 degrees, less than 2.5 degrees, or even lower with respect to an injection site or skin surface of a patient.

21. A medical syringe, comprising:
a substantially flat barrel comprising a main body portion that defines a fluid chamber, an annularly projecting finger flange disposed at a rear end of the main body portion, and a needle holder projecting forwardly from a nose end of the main body portion, wherein the substantially flat barrel comprises a width (W_{B}) and a thickness (T_{B}), and wherein the width is greater than the thickness;
a needle disposed in a needle aperture of the needle holder and projecting forwardly from the needle holder, wherein the needle is connected in fluid communication with the fluid chamber; and
a plunger assembly disposed within the substantially flat barrel and comprising a plunger surface for selectively actuating the plunger assembly, an elongated plunger body extending from the plunger surface, and a plunger seal disposed at a distal end of the elongated plunger body and configured to slidably engage the fluid chamber to form a fluid tight seal between the plunger assembly and the substantially flat barrel.

22. The medical syringe of claim 21, wherein the main body portion comprises substantially flat upper and lower surfaces.

23. The medical syringe of claim 21, wherein the elongated plunger body comprises a rib extending from the plunger surface and longitudinally along the elongated plunger body, and wherein the rib comprises a width (W_{R}) that is at least 10%, at least 15%, at least 20%, or at least 25% of a width (W_{P}) of the elongated plunger body.

24. The medical syringe of claim 21, wherein the substantially flat barrel comprises a width to thickness ratio (W_{B}:T_{B}) of at least 1.05:1 to not greater than 5:1, and wherein the fluid chamber comprises a width to thickness ratio (W_{FC}:T_{FC}) of at least 1.05:1 to not greater than 10:1.
